(19) United States
(12) Patent Application Publication
(10) Pub. No.: US 2012/0270793 A1
(43) Pub. Date: Oct. 25, 2012

(54) METHODS OF TREATING A PATIENT RECEIVING A CARDIAC STENT IMPLANT

(75) Inventor: Alexandra R. Lucas, Gainesville, FL (US)

(73) Assignee: Viron Therapeutics Inc., London, ON (CA)

(21) Appl. No.: 13/496,408

(22) PCT Filed: Oct. 21, 2010
(86) PCT No.: PCT/IB10/02701

§ 371 Date: Jun. 5, 2012

(51) Int. Cl. A61K 38/55; A61P 9/10; A61P 9/00

(52) U.S. Cl. 514/16.4

(57) ABSTRACT

Methods of treating a patient receiving a cardiac stent implant including the steps of: (i) monitoring the circulating level of Tn I or CK-MB in the patient; and (ii) administering SERP-I to the patient in an amount sufficient to prevent the circulating level of Tn I or CK-MB from exceeding a specified threshold for the first 24 hours following implantation are disclosed. A specified value of SERP-I is sufficient for preventing circulating level of Tn I or CK-MB from exceeding a specified threshold during the first 24 hours following implantation. The dosing regime of SERP-I to the patient starting within 24 hours of post-implantation is also disclosed.

### FIELD OF THE INVENTION

In general, this invention relates to methods of treating patients receiving a cardiac stent implant.

### BACKGROUND OF THE INVENTION

Placement of a cardiac stent implant frequently causes myocardial tissue damage, resulting in elevated levels of cardiac enzymes, e.g., cardiac Troponin I (TnI) and creatinine kinase MB fraction (CK-MB). The literature has established that there is a highly statistically significant correlation between circulating concentrations of these enzymes exceeding a predetermined threshold (e.g., TnI exceeding 0.5 or 0.8 ng/ml) in the first 24 hours following stent implantation, and the likelihood of a subsequent major adverse cardiac event (MACE). See, e.g., Cantor et al., J. Am. Coll. Cardiol. 39(11):1738-1744 (2002), and Ramirez-Moreno et al., Int. J. Cardiol. 97(2):193-198 (2004). There is a need in the art for methods of treating cardiac stent implant patients that reduce the likelihood of occurrence of MACE, e.g., by preventing the levels of cardiac enzymes such as TnI from exceeding predetermined thresholds.

### SUMMARY OF THE INVENTION

It has been discovered that administration of SERP-1 to a patient receiving a cardiac stent implant is effective in preventing the circulating levels of TnI and CK-MB from exceeding thresholds associated with increased likelihood of occurrence of a major adverse cardiac event (MACE). According to the methods of the invention, such administration of SERP-1 is useful, e.g., to reduce the likelihood of occurrence of a major adverse cardiac event (MACE) in a patient receiving a cardiac stent implant.

Accordingly, the invention features a method of treating a patient receiving a cardiac stent implant including the steps of (i) monitoring the circulating level of TnI in the patient; and (ii) administering SERP-1 to the patient in an amount sufficient to prevent the circulating level of TnI from exceeding a threshold of, e.g., 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, or 1.5 ng/ml for the first, e.g., 6 hours, 12 hours, 18 hours, 24 hours, 36 hours, 2 days, 3 days, or week following implantation of the stent in the patient. In some embodiments, the amount of SERP-1 administered to the patient is not more than, e.g., 1.5, 2, 2.5, 3, 3.5, 4, 4.5, 5, 6, 7, 8, 9, or 10 times the minimum dose of SERP-1 that is sufficient to prevent the circulating level of TnI from exceeding a specified threshold, e.g., 0.5 ng/ml, for the first, e.g., 24 hours following implantation of the stent in the patient. The first dose of SERP-1 may be provided prior to implantation of the stent.

The invention further features a method of treating a patient receiving a cardiac stent implant including the steps of: (i) monitoring the circulating level of TnI in the patient; and (ii) administering SERP-1 to the patient in an amount sufficient to achieve an exposure of SERP-1 of at least, e.g., 8.5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, or 500 ng·h/ml during the first, e.g., 6 hours, 12 hours, 18 hours, 24 hours, 36 hours, 2 days, 3 days, or week following implantation of the stent in the patient. In some embodiments, the amount of SERP-1 administered to the patient is not more than, e.g., 1.5, 2, 2.5, 3, 3.5, 4, 4.5, 5, 6, 7, 8, 9, or 10 times the minimum dose of SERP-1 that is sufficient to achieve at least a specified exposure of SERP-1, e.g., 8.5 ng·h/ml, during the first, e.g., 24 hours following implantation of the stent in the patient. The first dose of SERP-1 may be provided prior to implantation of the stent.

The invention further features a method of treating a patient receiving a cardiac stent implant including the steps of: (i) monitoring the circulating level of TnI in the patient; and (ii) administering SERP-1 to the patient in an amount of greater than, e.g., 0.5, 1.0, 2.5, 5, 10, 15, 20, 25, 30, 35, 40, 45, 50, 75, 100, 125, 150, 200, 250, 300, 400, or 500 μg/kg/day within, e.g., 6 hours, 12 hours, 18 hours, 24 hours, 36 hours, 2 days, 3 days, or one week of implantation of the stent in the patient. In some embodiments, the amount of SERF-1 is, e.g., from about 15 μg/kg/day to about 250 μg/kg/day, from about 15 μg/kg/day to about 150 μg/kg/day, from about 15 μg/kg/day to about 30 μg/kg/day, or about 15 μg/kg/day. The first dose of SERP-1 may be provided prior to implantation of the stent.

In any of the aforementioned methods featuring monitoring of TnI, step (i) may be performed subsequent to step (ii) and during the first, e.g., 6 hours, 12 hours, 18 hours, 24 hours, 36 hours, 2 days, 3 days, or week following stent implantation. Optionally, if the circulating level of TnI exceeds, e.g., 20%, 30%, 40%, 50%, 60%, 70%, 75%, 80%, 85%, 90%, 95%, 96%, 97%, 98%, or even 99% of the specified threshold of TnI, e.g., if the circulating level of TnI exceeds 0.40 ng/ml, which is 80% of the threshold value of 0.50 ng/ml, step (ii) may be repeated.

Preventing a level of the cardiac enzyme CK-MB from exceeding a specified threshold value may be an additional feature of the invention. In some embodiments, the foregoing methods may further comprise (iii) monitoring the circulating level of CK-MB in the patient; and/or (iv) administering SERP-1 to the patient in an amount sufficient to prevent the circulating level of CK-MB from exceeding a threshold of, e.g., 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, or 15 ng/ml for the first, e.g., 6 hours, 12 hours, 18 hours, 24 hours, 36 hours, 2 days, 3 days, or week following implantation of the stent in the patient.

The invention further features a method of treating a patient receiving a cardiac stent implant including the steps of: (i) monitoring the circulating level of CK-MB in the patient; and (ii) administering SERP-1 to the patient in an amount sufficient to prevent the circulating level of CK-MB from exceeding a threshold of, e.g., 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, or 15 ng/ml for the first, e.g., 6 hours, 12 hours, 18 hours, 24 hours, 36 hours, 2 days, 3 days, or week following implantation of the stent in the patient. In some embodiments, the amount of SERP-1 administered to the patient is not more than, e.g., 1.5, 2, 2.5, 3, 3.5, 4, 4.5, 5, 6, 7, 8, 9, or 10 times the minimum dose of SERP-1 that is sufficient to prevent the circulating level of CK-MB from exceeding a specified threshold, e.g., 5.0 ng/ml, for the first, e.g., 24 hours following implantation of the stent in the patient. The first dose of SERP-1 may be provided prior to implantation of the stent.

The invention further features a method of treating a patient receiving a cardiac stent implant including the steps of: (i) monitoring the circulating level of CK-MB in the patient; and (ii) administering SERP-1 to the patient in an amount sufficient to achieve an exposure of SERP-1 of at least, e.g., 8.5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, or 500 ng·h/ml during the first, e.g., 6 hours, 12 hours, 18 hours, 24 hours, 36 hours, 2 days, 3 days, or week following implantation of the stent in the patient. In some embodiments, the amount of SERP-1 administered to the patient is not more than, e.g., 1.5, 2, 2.5, 3, 3.5, 4, 4.5, 5, 6, 7, 8, 9, or 10 times the minimum dose of SERP-1 that is sufficient to achieve at least a specified exposure of SERP-1, e.g., 8.5 ng·h/ml, during the first, e.g., 24 hours following implantation of the stent in the patient. The first dose of SERP-1 may be provided prior to implantation of the stent.

The invention further features a method of treating a patient receiving a cardiac stent implant including the steps of: (i) monitoring the circulating level of CK-MB in the patient; and (ii) administering SERP-1 to the patient in an amount of greater than, e.g., 0.5, 1.0, 2.5, 5, 10, 15, 20, 25, 30, 35, 40, 45, 50, 75, 100, 125, 150, 200, 250, 300, 400, or 500 μg/kg/day within, e.g., 6 hours, 12 hours, 18 hours, 24 hours, 36 hours, 2 days, 3 days, or one week of implantation of the stent in the patient. In some embodiments, the amount of SERP-1 is, e.g., from about 15 μg/kg/day to about 250 μg/kg/day, from about 15 μg/kg/day to about 150 μg/kg/day, from about 15 μg/kg/day to about 30 μg/kg/day, or about 15 μg/kg/day.

In any of the aforementioned methods featuring monitoring of CK-MB, step (i) may be performed subsequent to step (ii) and during the first, e.g., 6 hours, 12 hours, 18 hours, 24 hours, 36 hours, 2 days, 3 days, or week following stent implantation, and if the circulating level of CK-MB exceeds, e.g., 20%, 30%, 40%, 50%, 60%, 70%, 75%, 80%, 85%, 90%, 95%, 96%, 97%, 98%, or even 99% of the specified threshold of CK-MB, e.g., if the circulating level of CK-MB exceeds 4.0 ng/ml, which is 80% of the threshold value of 5.0 ng/ml, step (ii) may be repeated.

In some embodiments, steps (i) and (ii) may be performed in either order or simultaneously and may be repeated once, twice, three times, four times, or more.

In some embodiments, SERP-1 may be administered prior to implantation of the stent in the patient, e.g., less than three days, two days, 24 hours, 18 hours, 12 hours, 6 hours, 5 hours, 4 hours, 3 hours, 2 hours, 1 hour, 30 minutes, 20 minutes, 10 minutes, 5 minutes, 1 minute, or less, prior to implantation of the stent in the patient.

In some embodiments, SERP-1 may be administered once about every, e.g., week, three days, two days, 24 hours, 18 hours, 12 hours, 6 hours, 5 hours, 4 hours, 3 hours, 2 hours, 1 hour, or 30 minutes, and may be administered over a period of, e.g., one day, two days, three days, four days, five days, six days, a week, two weeks, or even longer.

In some embodiments, SERP-1 is not administered for a second time during the first, e.g., week, three days, two days, 24 hours, 18 hours, 12 hours, 6 hours, 5 hours, 4 hours, 3 hours, 2 hours, 1 hour, or 30 minutes following implantation of the stent in the patient. For example, in some embodiments, SERP-1 may be administered only prior to implantation of the stent and not subsequently administered until after 24 hours following implantation of the stent.

In some embodiments, the stent is, e.g., a bare metal stent or a drug-eluting stent.

In some embodiments, SERP-1 is administered, e.g., intravenously.

In some embodiments, the amino acid sequence of SERP-1 includes, or consists of, an amino acid sequence that is at least, e.g., 50%, 60%, 70%, 75%, 80%, 85%, 90%, 95%, 96%, 97%, 98%, 99%, or even 100% identical to amino acids 16-369 of SEQ ID NO: 2, or a fragment or analog thereof having SERP-1 biological activity.

In some embodiments, SERP-1 is encoded by a nucleic acid molecule that hybridizes under high stringency conditions to at least a portion, e.g., to 20%, 30%, 40%, 50%, 60%, 70%, 75%, 80%, 85%, 90%, 95%, 96%, 97%, 98%, 99%, or even 100%, of a nucleic acid molecule including SEQ ID NO: 1.

In some embodiments, SERP-1 is at least, e.g., 50%, 60%, 70%, 75%, 80%, 85%, 90%, 95%, 96%, 97%, 98%, 99%, 99.5%, or 99.9% pure.

In some embodiments, SERP-1 is glycosylated.

In some embodiments, the patient is human.

In some embodiments, the methods described herein can reduce the likelihood of occurrence of a MACE for the first, e.g., 24 hours, two days, three days, week, two weeks, month, two months, three months, four months, five months, or six months following implantation of the stent in the patient. Only MACE events occurring within six months following stent implantation are considered for purposes of the present invention. In some embodiments, the methods of the invention can reduce the likelihood of occurrence of a MACE in the patient by at least, e.g., 10%, 20%, 25%, 30%, 35%, 40%, 45%, 50%, 55%, 60%, 65%, 70%, 75%, 80%, 85%, 90%, 95%, 99%, or more, within the first six months following stent implantation.

In some embodiments, the MACE is cardiovascular death, myocardial infarction, target lesion revascularization, e.g., including percutaneous coronary intervention (PCI), or coronary artery bypass graft (CABG).

In some embodiments, SERP-1 is formulated in a pharmaceutical composition that includes a pharmaceutically acceptable excipient.

The invention further features SERP-1 for use in a method of preventing the circulating level of Troponin I (Tne in a patient receiving a cardiac stent implant from exceeding a threshold of 0.5 ng/ml for the first 24 hours following implantation of the stent in the patient, wherein the method includes administering to the patient SERP-1 in an amount sufficient to prevent the circulating level of TnI from exceeding the threshold.

The invention further features SERP-1 for use in a method of preventing the circulating level of creatinine kinase MB fraction (CK-MB) in a patient receiving a cardiac stent implant from exceeding a threshold of 5.0 ng/ml for the first 24 hours following implantation of the stent in the patient, wherein the method includes administering to the patient SERP-1 in an amount sufficient to prevent the circulating level of CK-MB from exceeding the threshold.

The invention further features SERP-1 for use in a method of achieving an exposure of SERP-1 in a patient receiving a cardiac stent implant of at least 8.5 ng·h/ml during the first 24 hours following implantation of the stent in the patient, wherein the method includes administering to the patient SERP-1 in an amount sufficient to achieve the exposure of SERP-1.

The invention further features SERP-1 for use in a method of treating a patient receiving a cardiac stent implant, wherein the method includes administering SERP-1 to the patient in an amount of greater than 5 μg/kg/day within 24 hours of implantation of the stent in the patient.

The invention further features SERP-1 for use in a method of preventing the circulating level of Troponin I (TnI) in a patient receiving a cardiac stent implant from exceeding a threshold of 0.5 ng/ml for the first 24 hours following implantation of the stent in the patient, wherein the method includes administering a first dosage of SERP-1 to the patient prior to implantation of the stent in an amount sufficient to prevent the circulating level of TnI from exceeding the threshold.

The invention further features SERP-1 for use in a method of achieving an exposure of SERP-1 in a patient receiving a cardiac stent implant of at least 8.5 ng·h/ml during the first 24 hours following implantation of the stent in the patient, wherein the method includes administering a first dosage of SERP-1 to the patient prior to implantation of the stent in an amount sufficient to achieve the exposure of SERP-1.

The invention further features SERP-1 for use in a method of treating a patient receiving a cardiac stent implant, wherein the method includes administering a first dosage of SERP-1 to the patient prior to implantation of the stent in an amount of greater than 5 μg/kg/day within 24 hours of implantation of the stent in the patient.

Any and all methods described herein may be employed with any of the uses of SERP-1 described herein. In addition, SERP-1 may be used in the manufacture of a medicament in connection with any and all methods or uses described herein.

By “about” is meant ±10% of the recited value.

By “analog” in the context of SERP-1 is meant to include substitutions or alterations in the amino acid sequence of the SERP-1 polypeptide, which substitutions or alterations (e.g., additions and deletions) maintain at least one biological activity of the polypeptide, e.g., anti-inflammatory properties of the polypeptide when delivered to a site of inflammation, either directed at the site, i.e., locally, or systemically. The term “analog” includes amino acid insertional derivatives of SERP-1 such as amino and/or carboxylterminal fusions, as well as intrasequence insertions of single or multiple amino acids. Insertional amino acid sequence variants are those in which one or more amino acid residues are introduced into a predetermined site in the protein. Random insertion is also possible with suitable screening of the resulting product. Deletional variants are characterized by removal of one or more amino acids from the sequence. Substitutional amino acid variants are those in which at least one residue inserted in its place. Where the protein is derivatized by amino acid substitution, amino acids are generally replaced by other amino acids having similar physical chemical properties such as hydrophobicity, hydrophilicity, electronegativity, bulky sidechains and the like. Examples of conservative substitutions include the substitution of a non-polar (hydrophobic) residue such as isoleucine, valine, leucine or methionine for another. Likewise, the present invention contemplates the substitution of apolar (hydrophilic) residue such as between arginine and lysine, between glutamine and asparagine, and between glycine and serine. Additionally, the substitution of a basic residue such as lysine, arginine or histidine for another or the substitution of an acidic residue such as aspartic acid or glutamic acid for another is also contemplated. The term “analog” also encompasses homologs of SERP-1, e.g., corresponding amino acid sequences derived from other serpins and having the same or substantially the same biological activities or properties. For purposes of the present invention, analogs of SERP-1 also include single or multiple substitutions, deletions and/or additions of any component(s) naturally or artificially associated with the SERP-1 such as carbohydrate, lipid and/or other proteinaceous moieties. All such molecules are encompassed by the term “analog.”

By “an amount sufficient” in the context of administration of SERP-1 is meant the amount of SERP-1 required to treat or prevent in a clinically relevant manner. A sufficient amount of SERP-1 used to practice the present invention for therapeutic treatment of conditions caused by or contributing to a MACE varies depending upon the manner of administration, the age, body weight, and general health of the patient. Ultimately, the prescribers will decide the appropriate amount and dosage regimen.

The terms “circulating level” and “plasma concentration” are used interchangeably and refer to the concentration of a compound present in the plasma portion of the blood.

By “exposure” is meant the area under the curve (AUC_{0-∞}), as determined using standard pharmacokinetics analysis techniques.

By “fragment” is meant a portion of a polypeptide or nucleic acid molecule that contains, preferably, at least 10%, 20%, 30%, 40%, 50%, 60%, 70%, 75%, 80%, 85%, 90%, 95%, 96%, 97%, 98%, 99%, or more of the entire length of the reference nucleic acid molecule or polypeptide. A fragment may contain, e.g., 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 500, 600, 700, 800, 900, 1,000, 1,100, or more nucleotides, up to the entire length of the nucleic acid molecule, or 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 354, or more amino acids, up to the entire length of the polypeptide. Exemplary fragments of SERP-1 have SERP-1 biological activity, and may include, for example, all or a portion of residues 16-369 of SEQ ID NO: 2.

By “heterologous” is meant any two or more nucleic acid or polypeptide sequences that are not normally found in the same relationship to each other in nature. For instance, a heterologous nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

By “homolog” is meant a polypeptide or nucleic acid molecule exhibiting at least 50% identity to a reference amino acid sequence (e.g., SEQ ID NO: 2) or nucleic acid sequence (e.g., SEQ ID NO: 1). Such a sequence is generally at least, e.g., 50%, 60%, 70%, 75%, 80%, 85%, 90%, 95%, 96%, 97%, 98%, or 99% identical at the amino acid level or nucleic acid to a reference sequence. For polypeptides, the length of comparison sequences will generally be at least, e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 354, or more amino acids. For nucleic acids, the length of comparison sequences will generally be at least, e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 500, 600, 700, 800, 900, 1,000, 1,100, or more nucleotides.

By “hybridize” is meant to pair to form a double-stranded molecule between complementary polynucleotides, or portions thereof, under various conditions of stringency. (See, e.g., Wahl, G. M. and S. L. Berger (1987) Methods Enzymol. 152:399; Kimmel, A. R. (1987) Methods Enzymol. 152:507.) For example, high stringency salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. High stringency temperature conditions will ordinarily include temperatures of at least about 30° C., 37° C., or 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In an alternative embodiment, hybridization will occur at 37° C. in 500 mM NaCl, 50 mM trisodium citrate, 1% SDS, 35% formamide, and 100 μg/ml denatured salmon sperm DNA (ssDNA). In a further alternative embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, high stringency salt concentrations for the wash steps may be, e.g., less than about 30 mM NaCl and 3 mM trisodium citrate, or less than about 15 mM NaCl and 1.5 mM trisodium citrate. High stringency temperature conditions for the wash steps will ordinarily include a temperature of, e.g., at least about 25° C., 42° C., or 68° C. In one embodiment, wash steps will occur at 25° C. in 30 mM NaCl, 3 mM trisodium citrate, and 0.1% SDS. In an alternative embodiment, wash steps will occur at 42° C. in 15 mM NaCl, 1.5 mM trisodium citrate, and 0.1% SDS. In a further alternative embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “major adverse cardiac event” or “MACE” is meant cardiovascular death, myocardial infarction, target lesion revascularization, e.g., including percutaneous coronary intervention (PCI), or coronary artery bypass graft (CABG).

By “nucleic acid molecule” is meant a molecule, e.g., RNA or DNA, having a sequence of two or more covalently bonded, naturally occurring or modified nucleotides. The nucleic acid molecule may be, e.g., single or double stranded, and may include modified or unmodified nucleotides, or mixtures or combinations thereof. Various salts, mixed salts, and free acid forms are also included.

By “patient” or “subject” is meant a mammal, including, but not limited to, a human or non-human mammal, such as a bovine, equine, canine, ovine, or feline.

The terms “peptide,” “polypeptide,” and “protein” are used interchangeably and refer to any chain of two or more natural or unnatural amino acids, regardless of posttranslational modification (e.g., glycosylation or phosphorylation), constituting all or part of a naturally-occurring or non-naturally occurring polypeptide or peptide, as is described herein.

As used herein, a natural amino acid is a natural α-amino acid having the L-configuration, such as those normally occurring in natural polypeptides. Unnatural amino acid refers to an amino acid that normally does not occur in polypeptides, e.g., an epimer of a natural α-amino acid having the L configuration, that is to say an amino acid having the unnatural D-configuration; or a (D,L)-isomeric mixture thereof; or a homolog of such an amino acid, for example, a β-amino acid, an α,α-disubstituted amino acid, or an α-amino acid wherein the amino acid side chain has been shortened by one or two methylene groups or lengthened to up to 10 carbon atoms, such as an α-amino alkanoic acid with 5 up to and including 10 carbon atoms in a linear chain, an unsubstituted or substituted aromatic (α-aryl or α-aryl lower alkyl), for example, a substituted phenylalanine or phenylglycine.

By “pharmaceutically acceptable carrier” or “pharmaceutically acceptable excipient” is meant a carrier or excipient that is physiologically acceptable to the treated patient while retaining the therapeutic properties of the compound with which it is administered. One exemplary pharmaceutically acceptable carrier substance is physiological saline. Other physiologically acceptable carriers and their formulations are known to those skilled in the art and described, for example, in Remington's Pharmaceutical Sciences, (20^{th} edition), ed. A. Gennaro, 2000, Lippincott, Williams & Wilkins, Philadelphia, Pa.

By “pharmaceutical composition” is meant a composition containing SERP-1, formulated with a pharmaceutically acceptable excipient, and manufactured or sold with the approval of a governmental regulatory agency as part of a therapeutic regimen for the treatment or prevention of a disease or event in a mammal. Pharmaceutical compositions can be formulated, for example, for intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

By “purified” is meant separated from other naturally accompanying components. Typically, a compound (e.g., nucleic acid, polypeptide, or small molecule) is substantially pure when it is at least, e.g., 50%, 60%, 70%, 75%, 80%, 85%, 90%, 95%, 96%, 97%, 98%, 99%, 99.5%, or 99.9% by weight, free from proteins, antibodies, and naturally-occurring organic molecules with which it is naturally associated. In some instances, the factor is at least 99%, 99.5%, 99.9%, or even 99.99%, by weight, pure. A substantially pure factor may be obtained by chemical synthesis, separation of the factor from natural sources, or production of the factor in a recombinant host cell that does not naturally produce the factor. Proteins and small molecules may be purified by one skilled in the art using standard techniques such as those described by Ausubel et al. (Current Protocols in Molecular Biology, John Wiley & Sons, New York, 2000). The factor is preferably at least, e.g., 2, 5, or 10 times as pure as the starting material, as measured using polyacrylamide gel electrophoresis, column chromatography, optical density, HPLC analysis, or western analysis (Ausubel et al., supra). Preferred methods of purification include immunoprecipitation, column chromatography such as immunoaffinity chromatography, magnetic bead immunoaffinity purification, and panning with a plate-bound antibody.

By “SERP-1” is meant a polypeptide having an amino acid sequence that includes, or consists of, an amino acid sequence that is at least 50%, 60%, 70%, 75%, 80%, 85%, 90%, 95%, 96%, 97%, 98%, 99%, or even 100% identical to amino acids 1-369 or 16-369 of SEQ ID NO: 2, or a fragment or analog thereof having SERP-1 biological activity. For example, SERP-1 may have an amino acid sequence that consists of amino acids 16-369 of SEQ ID NO: 2, which constitutes the mature form of SERP-1 lacking the N-terminal signal sequence. Alternatively, SERP-1 may have an amino acid sequence that consists of amino acids 1-369 of SEQ ID NO: 2, which constitutes the immature form of SERP-1 that includes the N-terminal signal sequence. Also included are any derivatives of or modifications to a SERP-1 polypeptide, including but not limited to the modifications described herein. In one example, amino acids 1-15 of SEQ ID NO: 2 (the signal sequence) are modified or replaced to improve expression of SERP-1. In some embodiments, SERP-1 may be encoded by a nucleic acid molecule that hybridizes under high stringency conditions to at least a portion, e.g., to 20%, 30%, 40%, 50%, 60%, 70%, 75%, 80%, 85%, 90%, 95%, 96%, 97%, 98%, 99%, or even 100%, of a nucleic acid molecule that includes SEQ ID NO: 1.

SERP-1, a serine proteinase inhibitor originally identified from myxoma virus, is capable of inhibiting inflammation and atheroma development in rabbit and rat models after balloon injury and dramatically reducing macrophage invasion and atherosclerotic plaque growth in cholesterol fed rabbits after angioplasty injury (Lucas et al., Circulation 94:2890-2900 (1996)).

Preliminary studies in a rat aortic allograft model have also demonstrated significant reductions both in mononuclear cell invasion and transplant vasculopathy after infusion of SERP-1 (see, e.g., Miller et al., Circulation 101(13):1598-1605 (2000), which is hereby incorporated by reference).

In one embodiment, SERP-1 is a 55 kD glycoprotein that inhibits a variety of serine proteinases that regulate the inflammatory response. SERP-1 regulates thrombolytic proteins, plasmin, tissue plasminogen activator (tPA), and urokinase. A single local infusion of SERP-1 protein, cloned and expressed from a vaccinia vector, at the site of balloon injury, dramatically decreases subsequent plaque growth and macrophage invasion (see, e.g., Lucas, et al. (1996)). SERP-1 modulates transcription of elements of the thrombolytic cascade soon after endothelial injury. SERP-1 is the subject of numerous U.S. patents, including U.S. Pat. No. 5,686,409, entitled, “Antirestenosi's Protein”; U.S. Pat. Nos. 5,917,014 and 5,939,525, both entitled, “Methods of Treating Inflammation and Compositions Therefor”; U.S. Pat. No. 7,285,530, entitled, “Use of SERP-1 as an Antiplatelet Agent”; U.S. Pat. No. 7,419,670, entitled, “Method of Treating Arthritis with SERP-1 and an Immunosuppressant”; and U.S. Pat. No. 7,514,405, entitled, “Methods for Treating Transplant Rejection,” each of which is hereby incorporated by reference.

By “SERP-1 biological activity” is meant a biological property of the mature form of SERP-1 having residues 16-369 of SEQ ID NO: 2, including but not limited to antiinflammatory activity; anti-rejection activity in the context of organ transplantation; ability to treat platelet adhesion/aggregation or thrombus formation; or maintenance of, e.g., TnI or CK-MB below a specified threshold. Assays for SERP-1 activity are known in the art or are described herein.

By “SERP-1 nucleic acid molecule” is meant a nucleic acid molecule that encodes a SERP-1 polypeptide and that is at least, e.g., 50%, 55%, 60%, 65%, 70%, 75%, 80%, 85%, 90%, 95%, 96%, 97%, 98%, 99% or more identical to the nucleic acid sequence set forth in SEQ ID NO: 1. Also included in the definition are any nucleic acid molecules having a sequence that differs from SEQ ID NO:1 by substitution of a T with a U; nucleic acid molecules with sequences complementary to either the full length of SEQ ID NO:1, or complementary to nucleic acid fragments derived thereof; nucleic acid molecules that hybridize with nucleic acid sequences represented within SEQ ID NO:1; and nucleic acid molecules that have sequences differing from the full length of SEQ ID NO:1 due to the degeneracy of the genetic code.

By “signal sequence” is meant an amino acid sequence that directs a polypeptide to the cellular membrane such that the polypeptide is secreted. Alternatively, the signal sequence may direct the polypeptide to an intracellular compartment or organelle, such as the Golgi apparatus. A signal sequence may be identified by homology, or biological activity, to a peptide sequence with the known function of targeting a polypeptide to a particular region of the cell. One of ordinary skill in the art can identify a signal sequence by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). A signal sequence can be one that is, for example, substantially identical to amino acids 1-15 of SEQ ID NO: 2.

By “substantially identical” is meant a nucleic acid or amino acid sequence that, when optimally aligned, for example, using the methods described below, shares at least 50%, 55%, 60%, 65%, 70%, 75%, 80%, 85%, 90%, 95%, 96%, 97%, 98%, 99%, or even 100% sequence identity with a second nucleic acid or amino acid sequence, e.g., a SERP1 nucleic acid sequence or amino acid sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman J. Mol. Biol. 147:195-7, 1981); “BestFit” (Smith and Waterman, Advances in Applied Mathematics, 482-489, 1981) as incorporated into GeneMatcher Plus™, Schwarz and Dayhof “Atlas of Protein Sequence and Structure,” Dayhof, M. O., Ed pp 353-358, 1979; BLAST program (Basic Local Alignment Search Tool; (Altschul, S. F., W. Gish, et al., J. Mol. Biol. 215: 403-410, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. For polypeptides, the length of comparison sequences will generally be at least, e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 354, or more amino acids, or more up to the entire length of the polypeptide. For nucleic acids, the length of comparison sequences will generally be at least, e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 500, 600, 700, 800, 900, 1,000, 1,100, or more nucleotides, up to the entire length of the nucleic acid molecule. It is understood that, for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “subject” is meant a mammal, including, but not limited to, a human or nonhuman mammal, such as a monkey, rabbit, rat, bovine or equine.

By “sustained release” or “controlled release” is meant that SERP-1 is released from the formulation at a controlled rate such that therapeutically beneficial blood levels (but below toxic levels) of SERP-1 are maintained over an extended period of time ranging from e.g., about 12 to about 24 hours, thus, providing, for example, a 12 hour or a 24 hour dosage form.

By “treating” or “treatment” is meant the medical management of a patient with the intent to cure, ameliorate, stabilize, reduce the likelihood of, or prevent a disease, pathological condition, disorder, or event, e.g., a MACE, e.g., by administering a pharmaceutical composition. This term includes active treatment, that is, treatment directed specifically toward the improvement or associated with the cure of a disease, pathological condition, disorder, or event, and also includes causal treatment, that is, treatment directed toward removal of the cause of the associated disease, pathological condition, disorder, or event. In addition, this term includes palliative treatment, that is, treatment designed for the relief of symptoms rather than the curing of the disease, pathological condition, disorder, or event; symptomatic treatment, that is, treatment directed toward constitutional symptoms of the associated disease, pathological condition, disorder, or event; preventative treatment, that is, treatment directed to minimizing or partially or completely inhibiting the development of the associated disease, pathological condition, disorder, or event, e.g., in a patient who is not yet ill, but who is susceptible to, or otherwise at risk of, a particular disease, pathological condition, disorder, or event; and supportive treatment, that is, treatment employed to supplement another specific therapy directed toward the improvement of the associated disease, pathological condition, disorder, or event.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

By “within,” in the context of a temporal relationship, is meant before, during, or after the specified time window. For example, “within 24 hours” means at any time from 24 hours prior to 24 hours following a specified event.

Other features and advantages of the invention will be apparent from the detailed description and from the claims.

### BRIEF DESCRIPTION OF THE DRAWINGS

FIG. 1 is a listing of the nucleic acid sequence that encodes the immature form of myxoma virus SERP-1 as known in the art and deposited as GenBank Accession No. M35233.1 (SEQ ID NO: 1).

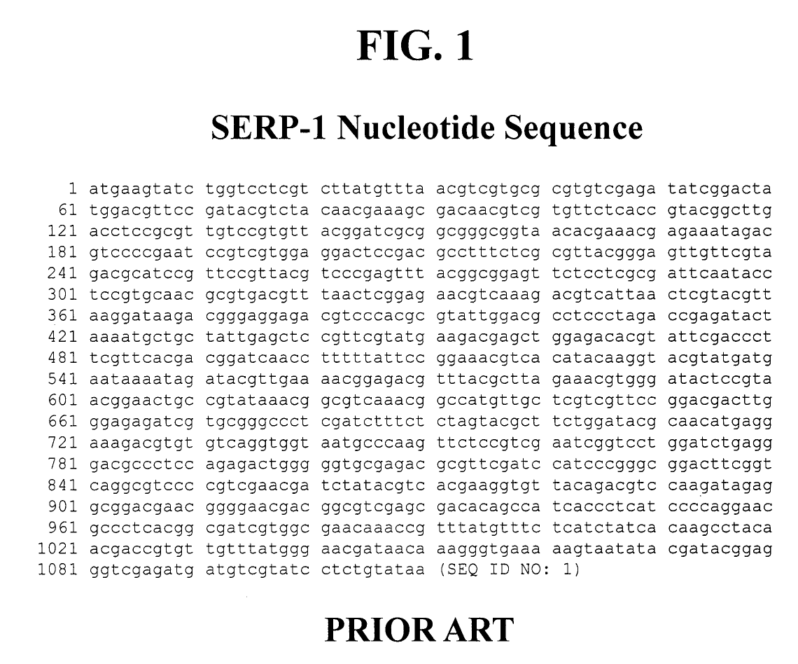

FIG. 2 is a listing of the amino acid sequence of the immature form of myxoma virus SERP-1, including the 15 amino acid N-terminal signal sequence, as known in the art and deposited as GenBank Accession No. AAA46629.1 (SEQ ID NO: 2). The mature form of SERP-1 spans amino acids 16-369 of SEQ ID NO: 2.

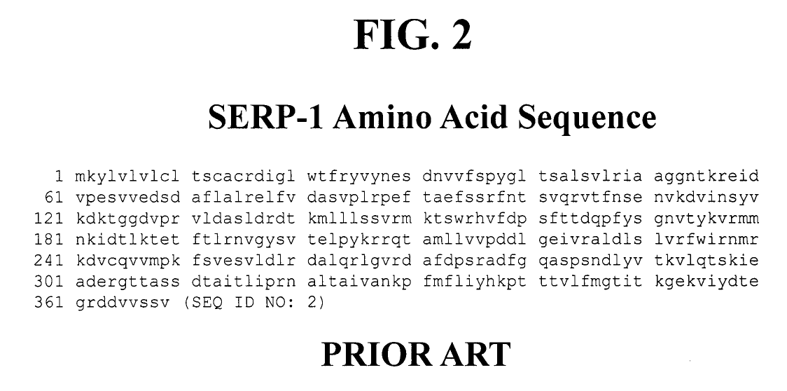

FIG. 3 is a graph that shows the plasma concentrations of TnI in acute coronary syndrome (ACS) patients. The effect of three daily IV injections of SERP-1, starting immediately prior to stent implantation, on TnI levels in ACS patients was measured at baseline, 8, 16, 24, 48, and 54 hours, and 14 and 28 days, post stent implantation. Adjusted geometric means are presented. *p<0.05, 15 μg/kg vs. placebo; **p<0.05 15 μg/kg vs. placebo; and 15 μg/kg vs. 5 μg/kg. The dotted line shows the threshold TnI level of 0.5 ng/ml.

FIG. 4 is a graph that shows the plasma concentrations of CK-MB in ACS patients. The effect of three daily IV injections of SERP-1, starting immediately prior to stent implantation, on CK-MB levels in ACS patients was measured at baseline, 8, 16, 24, 48, and 54 hours, and 14 and 28 days, post stent implantation. Adjusted geometric means are presented. *p<0.05, 15 μg/kg vs. placebo; **p<0.05 15 μg/kg vs. placebo; and 15 μg/kg vs. 5 μg/kg. The dotted line shows the threshold CK-MB level of 5.0 ng/ml.

### DETAILED DESCRIPTION OF THE INVENTION

The present invention features methods of treating a patient receiving a cardiac stent implant including the steps of: (i) monitoring the circulating level of TnI and/or CK-MB in the patient; and (ii) administering SERP-1 to the patient. In some instances, SERP-1 is administered in an amount sufficient to prevent the circulating level of TnI and/or CK-MB from exceeding a specified threshold, e.g. 0.5 ng/ml and/or 5.0 ng/ml, respectively, for the first, e.g., 24 hours following implantation of the stent in the patient, or is administered in an amount sufficient to achieve an exposure of SERP-1 of at least a specified value, e.g., 8.5 ng·h/ml, during the first, e.g., 24 hours following implantation of the stent in the patient, or is administered at a specified dosage level, e.g. from about 15 to about 250 μg/kg/day, within a specified time period, e.g., 24 hours following implantation of the stent in the patient, with the time period optionally starting from prior to implantation of the stent. As the results described herein demonstrate, such administration of SERP-1 is useful, e.g., to reduce the likelihood of occurrence of a major adverse cardiac event (MACE) in a patient receiving a cardiac stent implant.

Treatment

Treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the treatment depends on the age and condition of the patient, the nature of the cardiac stent implant, and how the patient responds to the treatment. Additionally, a person having a greater risk of developing a MACE may receive prophylactic treatment to inhibit, delay, or prevent it from occurring.

Formulation of Pharmaceutical Compositions

The pharmaceutical compositions of the invention are prepared in a manner known to those skilled in the art, for example, by means of conventional dissolving, lyophilising, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Suitable modes of administration include, but are not limited to, intravenous, parenteral, oral, subcutaneous, intramuscular, and transdermal.

Administration of compositions of the invention may be by any suitable means that results in a SERP-1 concentration that is effective for treating the patient. SERP-1 can be admixed with a suitable carrier substance, e.g., a pharmaceutically acceptable excipient that preserves the therapeutic properties of SERP-1. One exemplary pharmaceutically acceptable excipient is physiological saline. The suitable carrier substance is generally present in an amount of 0.1-99.9% by weight of the total weight of the composition. The composition may be provided in a dosage form that is suitable for intravenous, parenteral, oral, subcutaneous, intramuscular, or transdermal administration. Thus, the composition may be in the form of, e.g., intravenous fluid, tablets, capsules, pills, powders, granulates, suspensions, emulsions, solutions, gels including hydrogels, pastes, ointments, creams, plasters, delivery devices including pumps and coated stents, injectables, implants, etc.

Pharmaceutical compositions according to the invention may be formulated to release SERP-1 substantially immediately upon administration or at any predetermined time period after administration, using controlled release formulations.

SERP-1 may be expressed, e.g., as an immature polypeptide that includes a 15 amino acid N-terminal signal sequence and a 354 amino acid mature portion. The mature SERP-1 polypeptide may be obtained by co-or post-translational cleavage of the signal sequence or by other methods, e.g., recombinant methods. Exemplary nucleic acid and amino acid sequences for a SERP-1 nucleic acid and polypeptide are provided in SEQ ID NOs: 1 and 2, respectively. Thus, the signal sequence of SERP-1 corresponds to amino acids 1-15 of SEQ ID NO: 2, while the mature portion corresponds to amino acids 16-369 of SEQ ID NO: 2.

SERP-1 amino acid variants may readily be made using peptide synthetic techniques well known in the art such as solid phase peptide synthesis (Merrifield synthesis) and the like or by recombinant DNA techniques well known in the art. Manipulation of DNA sequences to produce substitutional, insertional, or deletional variants are conveniently described elsewhere such as Sambrook and Russell, 2001, Molecular Cloning: A Laboratory Manual, 3rd Ed., Cold Spring Harbor Laboratory Press, Cold Spring Harbor, N.Y.

Dosage

Appropriate dosages of SERP-1 used in the methods of the invention depend on several factors, including the route of administration, the severity of the patient's condition, and the age, weight, and health of the patient to be treated. Additionally, pharmacogenomic (the effect of genotype on the pharmacokinetic, pharmacodynamic or efficacy profile of a therapeutic) information about a particular patient may affect dosage used.

Continuous daily dosing with SERP-1 may not be required. A therapeutic regimen may require cycles, during which time SERP-1 is not administered, or therapy may be provided on an as-needed basis.

As described herein, SERP-1 is typically administered intravenously, though it may alternatively be administered parenterally, orally, subcutaneously, or by other routes. Appropriate SERP-1 dosages, e.g., for intravenous administration, according to the methods of the invention, include greater than, e.g., 0.5, 1.0, 2.5, 5, 10, 15, 20, 25, 30, 35, 40, 45, 50, 75, 100, 125, 150, 200, 250, 300, 400, or 500 μg/kg/day, or any range between such dosages, within, e.g., 24 hours of implantation of the stent in the patient. The SERP-1 may be first administered prior to implantation of the stent; optionally, the SERP-1 is not subsequently administered until after 24 hours following stent implantation. Dosages may be calculated based on the actual or approximate weight of the patient, or may be calculated based on a benchmark weight for a child or adult, e.g., 1, 2, 5, 10, 15, 20, 25, 30, 40, 50, 60, 70, 80, 90, 100, 125, 150, or even 200 kg. In some cases, the amount of SERP-1 is, e.g., from about 15 μg/kg/day to about 250 μg/kg/day, from about 15 μg/kg/day to about 150 μg/kg/day, from about 15 μg/kg/day to about 30 μg/kg/day, or about 15 μg/kg/day. SERP-1 may be administered once about every, e.g., week, three days, two days, 24 hours, 18 hours, 12 hours, 6 hours, 5 hours, 4 hours, 3 hours, 2 hours, 1 hour, or 30 minutes, and may be administered over a period of, e.g., one day, two days, three days, four days, five days, six days, a week, two weeks, or even longer.

In some instances, SERP-1 in, e.g., a 0.9% normal saline solution may be administered at dose levels of 5 μg/kg/day or 15 μg/kg/day by intravenous bolus injection daily for three days, with the first dose administered immediately prior to implantation of the stent in the patient, and subsequent doses given at about 24 and 48 hours later, respectively.

In some instances, SERP-1 is administered to the patient immediately prior to the stent implantation procedure so that there is already a circulating level of SERP-1 present within the patient's body before tissue damage occurs. In this manner, the levels of TnI and/or CK-MB may be prevented from exceeding their respective threshold values.

SERP-1 can also be administered in an amount sufficient to prevent the circulating level of TnI from exceeding a threshold of, e.g., 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, or 1.5 ng/ml for the first, e.g., 24 hours following implantation of the stent in the patient. In some cases, the amount of SERP-1 administered to the patient is not more than, e.g., 1.5, 2, 2.5, 3, 3.5, 4, 4.5, 5, 6, 7, 8, 9, or 10 times the minimum dose of SERF-1 that is sufficient to prevent the circulating level of TnI from exceeding a specified threshold, e.g., 0.5 ng/ml, for the first, e.g., 24 hours following implantation of the stent in the patient.

In addition, SERP-1 can be administered in an amount sufficient to achieve an exposure of SERP-1 of at least, e.g., 8.5, 10, 15, 20, 25, 30, 35, 40, 45, 50, 55, 60, 65, 70, 75, 80, 85, 90, 95, 100, 110, 120, 130, 140, 150, 160, 170; 180, 190, 200, 225, 250, 275, 300, 350, 400, or 500 ng·h/ml during the first, e.g., 24 hours following implantation of the stent in the patient. In some cases, the amount of SERF-1 administered to the patient is not more than, e.g., 1.5, 2, 2.5, 3, 3.5, 4, 4.5, 5, 6, 7, 8, 9, or 10 times the minimum dose of SERP-1 that is sufficient to achieve at least a specified exposure of SERP-1, e.g., 8.5 ng·h/ml, during the first, e.g., 24 hours following implantation of the stent in the patient.

SERP-1 can further be administered in an amount sufficient to prevent the circulating level of CK-MB from exceeding a threshold of, e.g., 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, or 15 ng/ml for the first, e.g., 24 hours following implantation of the stent in the patient. In some cases, the amount of SERP-1 administered to the patient is not more than, e.g., 1.5, 2, 2.5, 3, 3.5, 4, 4.5, 5, 6, 7, 8, 9, or 10 times the minimum dose of SERP-1 that is sufficient to prevent the circulating level of CK-MB from exceeding a specified threshold, e.g., 5.0 ng/ml, for the first, e.g., 24 hours following implantation of the stent in the patient.

For any route of administration of SERF-1, the above dosages may be appropriate, or other dosages in the range of about, e.g., 0.005 to 0.05 μg/kg/day, 0.05 to 0.5 μg/kg/day, 0.5 to 5.0 μg/kg/day, 5.0 to 50 μg/kg/day, 50 to 500 μg/kg/day, or 500 to 5,000 μg/kg/day, or any other range in between, may be used, provided that the threshold levels are not exceeded and/or the desired exposure level is reached.

SERP-1 may be administered, e.g., in a poorly sialylated form, e.g., as utilized in the Example described herein, or it may be administered in a more highly sialylated form. The glycosylation profile of SERP-1, in particular its sialylation profile, may have an effect on the appropriate dosage.

Monitoring of TnI and CK-MB

Any art-recognized method of monitoring the circulating levels of TnI and/or CKMB is appropriate in the methods described herein. Typically, the patient's blood is drawn at each time point, and the plasma concentration of TnI and/or CK-MB is determined using conventional assay methods known in the art. The monitoring step may be done before, during, and/or after implantation of the stent in the patient, and it also may be done before, during, and/or after administration of SERP-1 to the patient. Monitoring may be continuous or intermittent. A decision may be made to increase, decrease, or discontinue administration of SERP-1 based on the monitoring results. For example, if, after stent implantation, the circulating level of TnI is measured and found to exceed, e.g., 20%, 30%, 40%, 50%, 60%, 70%, 75%, 80%, 85%, 90%, 95%, 96%, 97%, 98%, or even 99% of the specified threshold of TnI, e.g., if the circulating level of TnI exceeds 0.40 ng/ml, which is 80% of the threshold value of 0.50 ng/ml, additional SERP1 may be administered. Likewise, if, after stent implantation, the circulating level of CKMB is measured and found to exceed, e.g., 20%, 30%, 40%, 50%, 60%, 70%, 75%, 80%, 85%, 90%, 95%, 96%, 97%, 98%, or even 99% of the specified threshold of CK-MB, e.g., if the circulating level of CK-MB exceeds 4.0 ng/ml, which is 80% of the threshold value of 5.0 ng/ml, additional SERP-1 may be administered.

In some embodiments, the monitoring step may be omitted.

### EXAMPLE

The following example is to illustrate the invention. It is not meant to limit the invention in any way.

Clinical Trial

A Phase II clinical trial was conducted from October, 2005, to December, 2008. The trial was labeled Serp-1-01-002 and entitled: “A Phase 2, Multicentre, Double-Blind, Placebo-Controlled, Dose Escalating Trial of the Safety, Pharmacokinetics, and Biological Activity of 3 Consecutive Daily Doses of Serp-1 When Added to Conventional Therapy in Patients With Acute Coronary. Syndromes (Non ST-Elevation Myocardial Infarction or Unstable Angina).”

The primary objective of this study was to evaluate the safety of SERP-1 injection when administered in three daily doses to patients undergoing conventional therapy for acute coronary syndromes (ACS) requiring early intervention. The patient population was males and females aged 18-80 years who presented with ACS (unstable angina or non ST-elevation MI), defined at a minimum as one or more episodes of angina lasting at least 5 minutes in the last 24 hours before admission and, per confirmatory angiogram, patient has been scheduled for percutaneous coronary angioplasty. Patients were enrolled into two cohorts (5 and 15 μg/kg SERP-1). A total of 48 patients were enrolled in the study, with 12 patients in the placebo group, 19 patients in the 5 μg/kg SERP-1 group, and 17 patients in the 15 μg/kg SERP-1 group.

Procedure

Upon enrollment, each subject remained at the study centre for the three days of the treatment period and then returned to the study centre at days 14, 28, and at 3 months and 6 months after treatment. Subjects received SERP-1 by intravenous (IV) bolus injection daily for 3 days, at dose levels of 5 or 15 μg/kg/dose (or placebo, 0.9% normal saline) added to the physician-prescribed therapy for ACS. SERP-1 was administered as a single IV bolus injection. The initial IV bolus dose was administered immediately preceding the PCI procedure for patients in whom an angiogram has been performed and urgent PCI was intended, with subsequent doses administered 24±2 and 48±2 hours later.

Dose levels were given sequentially in two cohorts. The second cohort of 15 μg/kg/dose was started following safety review of data from patients enrolled at the low dose (5.0 μg/kg/dose) level by the Data Safety Monitoring Board. The Data Safety Monitoring Board reviewed data gathered up to and including day 28 post-dose.

The following Clinical Procedures were followed during treatment:
Physical examination including vital signs, pulse, and blood pressure
Clinical laboratory evaluation (chemistry, hematology including coagulation parameters, urinalysis)
SERP-1 pharmacokinetic analysis
Inflammatory marker analysis
CK-MB and/or Troponin (T or I) release monitoring for 48 hours following admission

At days 14, and 28 following first dose, the subjects underwent the following safety evaluations:
Physical examination including vital signs, pulse, and blood pressure
Clinical laboratory evaluation (chemistry, hematology, coagulation parameters)
Inflammatory markers
CK-MB and/or Troponin (T or I) levels
12-lead ECG
Antibody analysis (also at 3 and 6 months)

The Safety Parameter Endpoints adopted for the study were as follows:
Significant changes in coagulation parameters
Immunogenicity of SERP-1 including neutralizing anti-SERP-1 and anti-PAI-1 antibodies
Significant changes in leukocyte count (neutropenia, leukopenia or signs of sepsis with febrile illness)
Allergic reactions

The Biologic Activity Endpoints adopted for the study were as follows:

Primary:
Decrease in levels of biochemical inflammatory markers (C-reactive protein, myeloperoxidase, myoglobin, D-dimer and BNP).

Secondary:
Rates of target vessel restenosis at 6 month follow-up using IVUS measurements.
Incidences of major adverse cardiac events (cardiovascular death, MI, or target lesion revascularization, e.g., intervention) through 6 months.

Since the goal of the study was to determine the safety of administering 3 daily doses of SERP-1 in this population, statistical analysis was performed to detect significant reductions in inflammatory markers, incidences of MACE, and restenosis rates.

Results:

Safety

There was no effect of SERP-1 on key safety endpoints, including coagulation parameters (prothombin time-international normalized ration (PT-INR) and activated partial thromboplastin time (aPTT)), leukocyte count (neutropenia, leucopenia, or signs of sepsis with febrile illness) or incidence of allergic reactions. Further, there was no effect of SERP-1 on vital signs, ECG and chest radiograph, or standard clinical laboratory evaluations (chemistry, hematology, urinalysis).

Antibodies

Plasma samples were analyzed at baseline, 14 and 28 days and 3 and 6 months following treatment for anti-SERP-1 and anti-PAI-1 antibodies. The overall false positive rates (study samples confirmed negative/overall study samples) for these analyses were 7.9% for the SERP-1 analysis and 4.7% for the PAI-1 analysis. The immunogenicity of SERP-1 was very low; one patient in the 5 μg/kg dose group was positive for anti-SERP-1 antibodies and three patients in the 15 μg/kg dose group were positive for anti-SERP-1 antibodies. Neutralizing activity was not detected in these patient samples. There were no anti-PAI antibodies detected in any patients.

Pharmacokinetics

The pharmacokinetics of SERP-1 was evaluated following each dose of SERP-1. The maximum plasma concentrations (Cₘₐₓ) of SERP-1 in Cohort 1 (5 μg/kg) subjects were between 7.13 and 203 ng/ml. The exposure, defined as the Area Under the Curve (AUC_{0-∞}), ranged between 9.92 and 82.3 ng·h/ml. In Cohort 2 (15 μg/kg) subjects, Cₘₐₓ ranged between 11.1 and 354 ng/ml. The exposure, defined as the Area Under the Curve (AUC_{0∞}), ranged between 18.3 and 76.7 ng·h/ml.

The systemic exposure of SERF-1 following three consecutive doses was variable across dose level and occasion. Gender differences in the pharmacokinetics of SERP-1 could not be concluded from this study.

Biomarker Analysis

The biological activity of SERP-1 was primarily measured by investigating the effect of SERF-1 on plasma biomarkers (PAI-1, MCP-1, MPO, CRP, D-dimer, Myoglobin, BNP, CK-MB and TNI) at several time points (baseline, 8, 16, 24, 48 and 54 hours and 14 and 28 days post-dose). The results of the analyses were analyzed by independent statisticians through repeated measures analysis of covariance (ANCOVA) models including a term for baseline value as well as terms for treatment, time and treatment×time interaction. A statistically significant treatment×time interaction suggests a possible treatment effect of SERP-1. Significant treatment×time interactions were following by treatment contracts under the repeated measures ANCOVA model, allowing for examination of treatment effect at each time point.

Prior to all analyses, the basic assumptions underlying the planned models were checked and log transformations were used to approximate normality for all biomarkers. All tests were two-sided and conducted at the 0.05 significance level. Analyses were performed by SAS release 9.1 (SAS Institute Inc. Cary, N.C., USA).

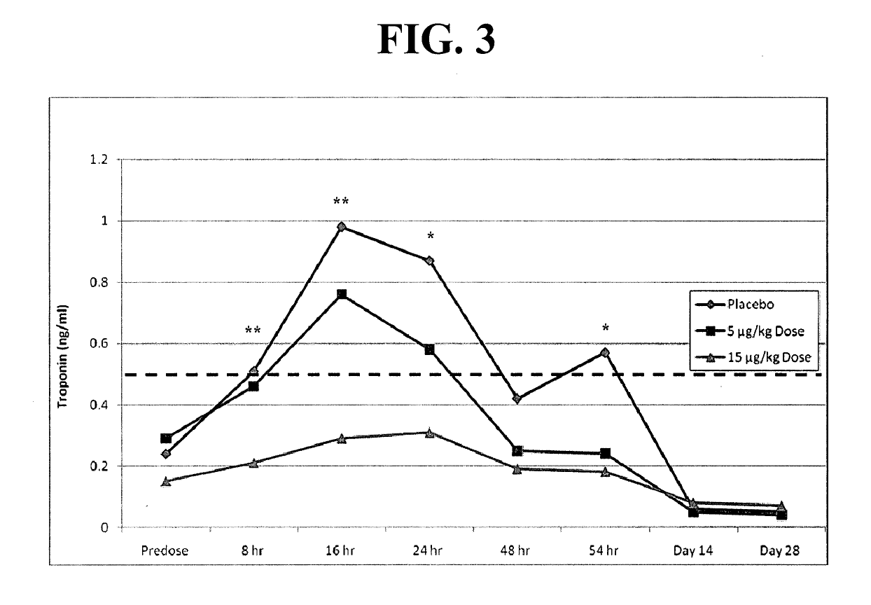

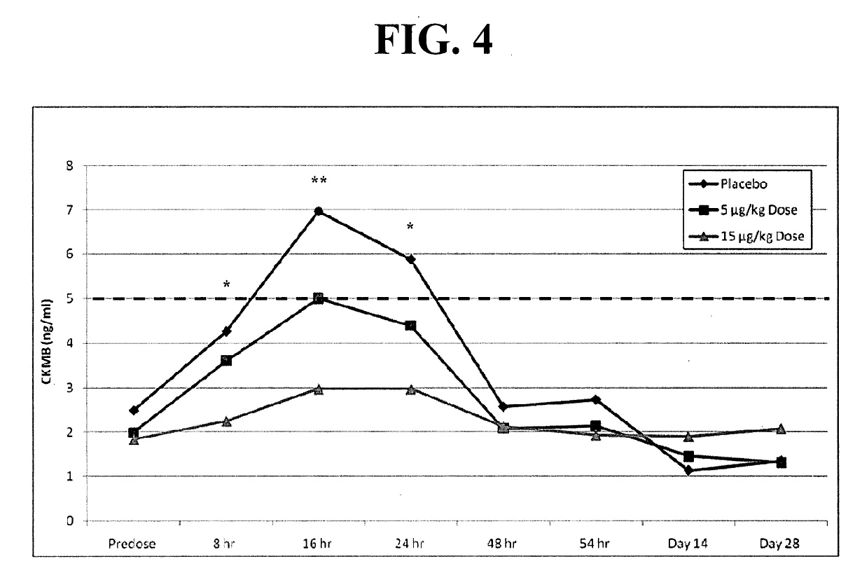

A significant dose-dependent effect was found for the key cardiac markers, TnI and CK-MB. A significant difference between placebo and 15 μg/kg and between 15 μg/kg and 5 μg/kg was observed at several time points (see Table 1 and FIGS. 3 and 4).

CK-MB has also been suggested in the literature to have a predictive effect on MACE events. SERP-1 had a dose-dependent effect on CK-MB, and the effect on this marker is supportive of the observations made with TnI.

TABLE 1
The effect of SERP-1 on cardiac enzymes in ACS patients.

| | P-value for | | | |
|---|---|---|---|---|
| | treatment × | | Adjusted | |
| Biomarker | time interaction | Time | geometric means | P-value* |
| TnI | 0.0191 | Baseline | | NS |
| | | 8 h | 0.21 (15 μg/kg) vs. | 0.0455 |
| | | | 0.46 (5 μg/kg) | |
| | | | 0.21 (15 μg/kg) vs | 0.0493 |
| | | | 0.51 (placebo) | |
| | | 16 h | 0.29 (1 μg/kg) vs. | 0.0158 |
| | | | 0.76 (5 μg/kg) | |
| | | | 0.29 (15 μg/kg) vs. | 0.0072 |
| | | | 0.98 (placebo) | |
| | | 24 h | 0.31 (15 μg/kg) vs. | 0.0237 |
| | | | 0.87 (placebo) | |
| | | 48 h | | NS |
| | | 54 h | 0.18 (15 μg/kg) vs. | 0.0153 |
| | | | 0.57 (placebo) | |
| | | 14 day | | NS |
| | | 28 day | | NS |
| CK-MB | 0.0090 | Baseline | | NS |
| | | 8 h | 2.25 (15 μg/kg) vs. | 0.0420 |
| | | | 4.26 (placebo) | |
| | | 16 h | 2.97 (15 μg/kg) vs. | 0.0338 |
| | | | 5.00 (5 μg/kg) | |
| | | | 2.97 (15 μg/kg) vs. | 0.0071 |
| | | | 6.96 (placebo) | |
| | | 24 h | 2.97 (15 μg/kg) vs. | 0.0339 |
| | | | 5.87 (placebo) | |
| | | 48 h | | NS |
| | | 54 h | | NS |
| | | 14 day | | NS |
| | | 28 day | | NS |

NS: Not statistically significant at the 0.05 level.
*P-values for the comparison between treatment groups

Rates of Target Vessel Restenosis at 6 Months:

The effect of SERP-1 on rates of target vessel restenosis was evaluated by Intravascular Ultrasound (IVUS) evaluations at baseline and 6 months. IVUS parameters were assessed in the lesion as well as in a reference segment.

The following parameters were evaluated:
In the lesion (stented area):
Lumen volume, stent volume and in-stent neointimal volume indexed for a 15 mm lesion by 3D IVUS;
Lumen area, stent area and intimal plaque area by 2D IVUS at the stent site with the smallest lumen area at follow-up;
Mean lumen area, mean stent are and mean neointimal area over the entire analyzed segment by 3D IVUS

In the reference segments:
Lumen volume, external elastic membrane (vessel) volume and plaque volume indexed for a 10 mm segment by 3D IVUS
There were no statistically significant differences detected in in-stent plaque or lumen area between control and treated groups.

Incidences of Major Cardiac Events:

The incidence of major adverse cardiac events (MACE) was captured for all “intent-to-treat” patients through to the final 6-month follow-up visit. The “intent-to-treat” population was a subset of the safety population, which included all randomized patients who took any dose of study drug (or placebo) during the study, and who had measurements of inflammatory biomarkers at both baseline and post-baseline timepoints. MACE was defined as cardiovascular death, myocardial infarction (MI), target lesion revascularization, or CABG.

Referring to Table 2, none of the 17 patients receiving the 15 μg/kg dose of SERP-1 had demonstrated incidences of MACE by the 6-month follow-up, while 7 out of 28 patients receiving either placebo or the 5 μg/kg dose demonstrated incidences of MACE by the 6-month follow-up. These results are consistent with the observed efficacy of SERP-1 in preventing TnI and CK-MB from exceeding their respective threshold values following stent implantation. In particular, as shown in FIG. 3, on average, patients receiving the higher SERP-1 dose were able to maintain a plasma concentration of TnI substantially below the threshold of 0.5 ng/ml throughout the critical 24-hour period following stent implant, while patients receiving placebo or the lower SERP-1 dose were not. Likewise, FIG. 4 shows that, on average, patients receiving the higher SERP-1 dose were able to maintain a plasma concentration of CK-MB substantially below the threshold of 5.0 ng/ml throughout the critical 24-hour period following stent implant, while patients receiving placebo or the lower SERP-1 dose were not.

TABLE 2
Incidences of Major Adverse Cardiac Events (Intent-to-treat population)

| | Placebo | 5.0 μg | 15 μg | |
|---|---|---|---|---|
| | (N = 11) | (N = 17) | (N = 17) | P Value [1] |
| Incidences of MACE | | | | 0.0471 |
| Yes | 2 (18.2%) | 5 (29.4%) | 0 | |
| No | 9 (81.8%) | 12 (70.6%) | 17 (100.0%) | |
| Total | 11 | 17 | 17 | |
| P-Value vs. Placebo [2] | | 0.6683 | 0.1455 | |
| Incidences of Myocardial Infarction | | | | 0.0993 |
| Yes | 1 (9.1%) | 4 (23.5%) | 0 | |
| No | 10 (90.9%) | 13 (76.5%) | 17 (100.0%) | |
| Total | 11 | 17 | 17 | |
| P-Value vs. Placebo [2] | | 0.6195 | 0.3929 | |
| Incidences of a Repeat PCI of Target Lesion(s) | | | | 0.2726 |
| Yes | 1 (9.1%) | 3 (17.6%) | 0 | |
| No | 10 (90.9%) | 14 (82.4%) | 17 (100.0%) | |
| Total | 11 | 17 | 17 | |
| P-Value vs. Placebo [2] | | 1.0000 | 0.3929 | |
| Incidences of Bypass Surgery of Target Vessels | | | | N/A |
| Yes | 0 | 0 | 0 | |
| No | 11 (100.0%) | 17 (100.0%) | 17 (100.0%) | |
| Total | 11 | 17 | 17 | |
| P-Value vs. Placebo [2] | | N/A | N/A | |
| Incidences of Cardiovascular Death | | | | N/A |
| Yes | 0 | 0 | 0 | |
| No | 11 (100.0%) | 17 (100.0%) | 17 (100.0%) | |
| Total | 11 | 17 | 17 | |
| P-Value vs. Placebo [2] | | N/A | N/A | |

Note:
Major adverse cardiac events (MACE) is defined as cardiovascular death, MI, target lesion revascularization, or CABG.
Subjects may have more than one MACE.
[1] Fisher's Exact Test across all treatments.
[2] Fisher's Exact Test, without adjustment for multiple comparisons.

Table 3 summarizes the results shown in Table 2, but with N values and percentages calculated for patients in the safety population.

TABLE 3
Incidences of Major Adverse Cardiac Events (Safety Population)

| | Placebo | 5 μg/kg | 15 μg/kg |
|---|---|---|---|
| | (n = 12) | (n = 19) | (n = 17) |
| Cardiovascular Death | 0 | 0 | 0 |
| Myocardial Infarction (# events) | 1 | 4 | 0 |
| Repeat PCI (# events) | 1 | 3 | 0 |
| CABG | 0 | 0 | 0 |
| Total MACE (# patients) | 2 (17%) | 5 (26%) | 0 (0%) |

Discussion

The biological activity of SERP-1 was measured by investigating the effect of SERP-1 on plasma biomarkers, including key markers of cardiac damage, TnI and CK-MB. A statistically significant, dose-dependent effect was observed for TnI and CK-MB in the first 24 hours following stent implant and SERP-1 administration. Furthermore, none of the high-dose patients had demonstrated incidences of MACE by the 6-month follow-up, while approximately a quarter of the placebo/low-dose patients demonstrated such incidences. The difference in MACE incidence between the higher-dose group on the one hand, and the placebo/lower-dose group on the other, demonstrates the efficacy of administering, e.g., 15 μg/kg SERP-1 not only to prevent the circulating levels of TnI and CK-MB from exceeding their respective threshold values, but also to reduce significantly the likelihood of a MACE following a stent implant.

Several clinical studies suggest that elevated TnI and/or CK-MB post-stent implant is significantly related to MACE events, including myocardial infarction and cardiovascular death (Cantor et al., 2002, Ramirez-Moreno et al., 2004). The cut-off level of TnI in patients for this relation has been reported as being between 0.5 and 1.5 ng/ml, with some studies suggesting that a range of 0.5 and 0.8 ng/ml may be more reflective of the threshold level of TnI required for predictability of increased MACE events. Similarly, the threshold for a relation between CK-MB and adverse events appears to be approximately 5.0 ng/ml.

In this study, a dose-dependent decrease of TnI and CK-MB following SERP-1 administration was observed. A reduction in MACE events was also observed. Doses of SERP-1 that prevented the level of TnI from exceeding 0.5 ng/mL and/or prevented the level of CK-MB from exceeding 5 ng/mL were found to have the greatest effect on MACE.

Given the relatively small patient population in the trial as described herein, it is surprising that any effect at all was observed. It is even more surprising that SERP-1 had a dose dependent impact on the cardiac enzymes TnI and CK-MB. While the literature predicts that there is a threshold level for TnI and CK-MB that, if exceeded, is predictive of MACE, it was not apparent that administration of SERP-1 would be able to prevent the levels from exceeding this threshold, nor that administration of SERP-1 would have a statistically significant effect on MACE. The fact that administration of SERP-1 in patients receiving a stent implant was able to dramatically reduce the incidence of MACE to zero was particularly striking. These observations of the effect of SERP-1 on MACE have the potential to significantly improve the outcome of stent implantation procedures and save the lives of patients who might otherwise die of a heart attack following the procedure.

### REFERENCES

Cantor W, Newby L, Christenson R, Tuttle R, Hasselblad V, Armstrong P, Moliterno D, Califf R, Topol E, Ohman E. Prognostic significance of elevated troponin I after percutaneous coronary intervention. J. Am. Coll. Cardiol. 2002; 39:1738-1744.
Davies B. and Morris T. Physiological parameters in laboratory animals and humans. Pharm. Res. 1993; 10:1093-1095.
Jiang J, Arp J, Kubelik D, Zassoko R, Liu W, Wise Y, Macaulay C, Garcia B, McFadden G, Lucas A, Wang H. Induction of indefinite cardiac allograft survival correlates with toll-like receptor 2 and 4 downregulation after serine protease inhibitor-1 (SERP-1) treatment. Transplantation 2007; 84:1158-1167.
Lucas A, Dai E, Liu L, Guan H, Nash P, McFadden G, Miller L. Transplant vasculopathy: viral anti-inflammatory serpin regulation of atherogenesis. J Heart Lung Transplant 2000; 19:1029-1038.
Lucas A, Liu L, Macen J, Nash P, Dai E, Stewart M, Graham K, Etches W, Boshkov L, Nation P, Humen D, Hobman M, McFadden G. Virus-encoded serine proteinase inhibitor SERP-1 inhibits atherosclerotic plaque development after balloon angioplasty. Circulation 1996; 94:2890-2900.
Miller L, Dai E, Nash P, Liu L, Icton C, Klironomos D, Fan L, Nation P, Zhong R, McFadden G, Lucas A. Inhibition of transplant vasculopathy in a rat aortic allograft model after infusion of anti-inflammatory viral serpin. Circulation. 2000; 101:1598-1605.
Ramirez-Moreno A, Cardenal R, Pera C, Pagola C, Guzman M, Vazquez E, Fajardo A, Lozano C, Solis J, Gasso M. Predictors and prognostic value of myocardial injury following stent implantation. Int. J. Cardiol. 2004; 97:194-198.

### Other Embodiments

All publications, patents, and patent applications mentioned in the above specification are hereby incorporated by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of treating a patient receiving a cardiac stent implant comprising the steps of:
(i) monitoring the circulating level of Troponin I (TnI) in said patient; and
(ii) administering SERP-1 to said patient in an amount sufficient to prevent said circulating level of TnI from exceeding a threshold of 0.5 ng/ml for the first 24 hours following implantation of said stent in said patient.

2. The method of claim 1, wherein said amount of said SERP-1 is not more than three times the minimum dose of SERP-1 that is sufficient to prevent said circulating level of TnI from exceeding a threshold of 0.5 ng/ml for the first 24 hours following implantation of said stent in said patient.

3. A method of treating a patient receiving a cardiac stent implant comprising the steps of:
(i) monitoring the circulating level of TnI in said patient; and
(ii) administering SERP-1 to said patient in an amount sufficient to achieve an exposure of SERP-1 of at least 8.5 ng·h/ml during the first 24 hours following implantation of said stent in said patient.

4. The method of claim 3, wherein said amount of said SERP-1 is not more than three times the minimum dose of SERP-1 that is sufficient to achieve the exposure of SERP-1 of at least 8.5 ng·h/ml during the first 24 hours following implantation of said stent in said patient.

5. A method of treating a patient receiving a cardiac stent implant comprising the steps of:
(i) monitoring the circulating level of TnI in said patient; and
(ii) administering SERP-1 to said patient in an amount of greater than 5 μg/kg/day within 24 hours of implantation of said stent in said patient.

6. The method of claim 5, wherein said amount of said SERP-1 is from about 15 μg/kg/day to about 250 μg/kg/day.

7. The method of claim 6, wherein said amount of said SERP-1 is from about 15 μg/kg/day to about 150 μg/kg/day.

8. The method of claim 7, wherein said amount of said SERP-1 is about 15 μg/kg/day.

9. The method of claim 1, wherein step (i) is performed subsequent to step (ii) during the first 24 hours following stent implantation, and wherein, if said circulating level of TnI exceeds 0.40 ng/ml, step (ii) is repeated.

10. A method of treating a patient receiving a cardiac stent implant comprising the steps of:
(i) monitoring the circulating level of creatinine kinase MB fraction (CKMB) in said patient; and
(ii) administering SERP-1 to said patient in an amount sufficient to prevent said circulating level of CK-MB from exceeding a threshold of 5.0 ng/ml for the first 24 hours following implantation of said stent in said patient.

11. The method of claim 10, wherein said amount of said SERP-1 is not more than three times the minimum dose of SERP-1 that is sufficient to prevent said circulating level of CK-MB from exceeding a threshold of 5.0 ng/ml for the first 24 hours following implantation of said stent in said patient.

12. A method of treating a patient receiving a cardiac stent implant comprising the steps of:
(i) monitoring the circulating level of CK-MB in said patient; and
(ii) administering SERP-1 to said patient in an amount sufficient to achieve an exposure of SERP-1 of at least 8.5 ng·h/ml during the first 24 hours following implantation of said stent in said patient.

13. The method of claim 12, wherein said amount of said SERP-1 is not more than three times the minimum dose of SERP-1 that is sufficient to achieve the exposure of SERP-1 of at least 8.5 ng·h/ml during the first 24 hours following implantation of said stent in said patient.

14. A method of treating a patient receiving a cardiac stent implant comprising the steps of:
(i) monitoring the circulating level of CK-MB in said patient; and
(ii) administering SERP-1 to said patient in an amount of greater than 5 μg/kg/day within 24 hours of implantation of said stent in said patient.

15. The method of claim 14, wherein said amount of said SERP-1 is from about 15 μg/kg/day to about 250 μg/kg/day.

16. The method of claim 15, wherein said amount of said SERP-1 is from about 15 μg/kg/day to about 150 μg/kg/day.

17. The method of claim 16, wherein said amount of said SERP-1 is about 15 μg/kg/day.

18. The method of claim 10, wherein step (i) is performed subsequent to step (ii) and during the first 24 hours following stent implantation, and wherein, if said circulating level of CK-MB exceeds 4.0 ng/ml, step (ii) is repeated.

19. The method of claim 1, wherein said SERP-1 is administered prior to implantation of said stent in said patient.

20. The method of claim 19, wherein said SERP-1 is administered less than 6 hours prior to implantation of said stent in said patient.

21. The method of claim 19, wherein SERP-1 is not administered during the first 24 hours following implantation of said stent in said patient.

22. The method of claim 1, wherein said SERP-1 is administered once about every 24 hours.

23. The method of claim 22, wherein said SERF-1 is administered once about every 24 hours for three days.

24. The method of claim 1, wherein said stent is a bare metal stent.

25. The method of claim 1, wherein said stent is a drug-eluting stent.

26. The method of claim 1, wherein said SERP-1 is administered intravenously.

27. The method of claim 1, wherein said SERP-1 comprises an amino acid sequence that is at least 90% identical to amino acids 16-369 of SEQ ID NO: 2.

28. The method of claim 27, wherein said SERP-1 comprises an amino acid sequence that is at least 95% identical to amino acids 16-369 of SEQ ID NO: 2.

29. The method of claim 28, wherein said SERP-1 comprises amino acids 16-369 of SEQ ID NO: 2.

30. The method of claim 27, wherein the amino acid sequence of said SERP-1 consists of an amino acid sequence that is at least 90% identical to amino acids 16-369 of SEQ ID NO: 2.

31. The method of claim 30, wherein the amino acid sequence of said SERP-1 consists of an amino acid sequence that is at least 95% identical to amino acids 16-369 of SEQ ID NO: 2.

32. The method of claim 31, wherein the amino acid sequence of said SERP-1 consists of amino acids 16-369 of SEQ ID NO: 2.

33. The method of claim 1, wherein said SERP-1 is encoded by a nucleic acid molecule that hybridizes under high stringency conditions to at least a portion of a nucleic acid molecule comprising SEQ ID NO: 1.

34. The method of claim 1, wherein said SERP-1 is at least 90% pure.

35. The method of claim 34, wherein said SERP-1 is at least 99% pure.

36. The method of claim 1, wherein said SERP-1 is glycosylated.

37. The method of claim 1, wherein said patient is human.

38. The method of claim 1, wherein said method reduces the likelihood of occurrence of a major adverse cardiac event (MACE) for the first 6 months following implantation of said stent in said patient.

39. The method of claim 38, wherein said method reduces the likelihood of occurrence of a MACE in said patient by at least 25%.

40. The method of claim 38, wherein said MACE is cardiovascular death.

41. The method of claim 38, wherein said MACE is myocardial infarction.

42. The method of claim 38, wherein said MACE is target lesion revascularization.

43. The method of claim 42, wherein said target lesion revascularization comprises percutaneous coronary intervention.

44. The method of claim 38, wherein said MACE is coronary artery bypass graft (CABG).

45. The method of claim 1, wherein said SERP-1 is formulated in a pharmaceutical composition comprising a pharmaceutically acceptable excipient.

46. A method of treating a patient receiving a cardiac stent implant comprising the steps of:
(i) monitoring the circulating level of Troponin I (TnI) in said patient; and
(ii) administering a first dosage of SERP-1 to said patient prior to implantation of said stent in an amount sufficient to prevent said circulating level of TnI from exceeding a threshold of 0.5 ng/ml for the first 24 hours following implantation of said stent in said patient.

47. A method of treating a patient receiving a cardiac stent implant comprising the steps of:
(i) monitoring the circulating level of TnI in said patient; and
(ii) administering a first dosage of SERP-1 to said patient prior to implantation of said stent in an amount sufficient to achieve an exposure of SERP 1 of at least 8.5 ng·h/ml during the first 24 hours following implantation of said stent in said patient.

48. A method of treating a patient receiving a cardiac stent implant comprising the steps of:
(i) monitoring the circulating level of TnI in said patient; and
(ii) administering a first dosage of SERP-1 to said patient prior to implantation of said stent in an amount of greater than 5 μg/kg/day.

49. The method of claim 46, wherein said method reduces the likelihood of occurrence of a major adverse cardiac event (MACE) for the first 6 months following implantation of said stent in said patient.

50. The method of claim 46, wherein said method further comprises administering a second dosage of said SERP-1 to said patient after 24 hours following implantation of said stent in said patient.

51-103. (canceled)


SEQUENCE LISTING (2 sequences)

SEQ ID NO: 1 - DNA, 1110 bp, Myxoma virus
atgaagtatctggtcctcgtcttatgtttaacgtcgtgcgcgtgtcgagatatcggactatggacgttccgatacgtctacaacgaaagcgacaacgtcgtgttctcaccgtacggcttgacctccgcgttgtccgtgttacggatcgcggcgggcggtaacacgaaacgagaaatagacgtccccgaatccgtcgtggaggactccgacgcctttctcgcgttacgggagttgttcgtagacgcatccgttccgttacgtcccgagtttacggcggagttctcctcgcgattcaatacctccgtgcaacgcgtgacgtttaactcggagaacgtcaaagacgtcattaactcgtacgttaaggataagacgggaggagacgtcccacgcgtattggacgcctccctagaccgagatactaaaatgctgctattgagctccgttcgtatgaagacgagctggagacacgtattcgacccttcgttcacgacggatcaacctttttattccggaaacgtcacatacaaggtacgtatgatgaataaaatagatacgttgaaaacggagacgtttacgcttagaaacgtgggatactccgtaacggaactgccgtataaacggcgtcaaacggccatgttgctcgtcgttccggacgacttgggagagatcgtgcgggccctcgatctttctctagtacgcttctggatacgcaacatgaggaaagacgtgtgtcaggtggtaatgcccaagttctccgtcgaatcggtcctggatctgagggacgccctccagagactgggggtgcgagacgcgttcgatccatcccgggcggacttcggtcaggcgtccccgtcgaacgatctatacgtcacgaaggtgttacagacgtccaagatagaggcggacgaacggggaacgacggcgtcgagcgacacagccatcaccctcatccccaggaacgccctcacggcgatcgtggcgaacaaaccgtttatgtttctcatctatcacaagcctacaacgaccgtgttgtttatgggaacgataacaaagggtgaaaaagtaatatacgatacggagggtcgagatgatgtcgtatcctctgtataa

SEQ ID NO: 2 - protein, 369 aa, Myxoma virus
MKYLVLVLCLTSCACRDIGLWTFRYVYNESDNVVFSPYGLTSALSVLRIAAGGNTKREIDVPESVVEDSDAFLALRELFVDASVPLRPEFTAEFSSRFNTSVQRVTFNSENVKDVINSYVKDKTGGDVPRVLDASLDRDTKMLLLSSVRMKTSWRHVFDPSFTTDQPFYSGNVTYKVRMMNKIDTLKTETFTLRNVGYSVTELPYKRRQTAMLLVVPDDLGEIVRALDLSLVRFWIRNMRKDVCQVVMPKFSVESVLDLRDALQRLGVRDAFDPSRADFGQASPSNDLYVTKVLQTSKIEADERGTTASSDTAITLIPRNALTAIVANKPFMFLIYHKPTTTVLFMGTITKGEKVIYDTEGRDDVVSSV